(19) United States
(12) Patent Application Publication
(10) Pub. No.: US 2025/0163511 A1
(43) Pub. Date: May 22, 2025

(54) MICRO RNA BIOMARKERS FOR THE DIAGNOSIS OF USHER SYNDROME

(71) Applicant: Father Flanagan's Boys' Home Doing Business as Boys Town National Research Hospital, Omaha, NE (US)

(72) Inventor: M. Rohan Fernando, Omaha, NE (US)

(21) Appl. No.: 18/949,325

(22) Filed: Nov. 15, 2024

Related U.S. Application Data
(60) Provisional application No. 63/599,660, filed on Nov. 16, 2023.

(51) Int. Cl. C12Q 1/6883

(52) U.S. Cl. CPC C12Q 1/6883; C12Q 2600/158; C12Q 2600/178

(57) ABSTRACT

Micro RNA (miRNA) biomarkers for the detection, diagnosis, and typing of Usher Syndrome are provided, as are related methods and kits. The methods can comprise detecting differential expression of one or more miRNA associated with Usher Syndrome in a patient, wherein the miRNA is differentially expressed as compared to expression of the same miRNA in a control cell line.

### CROSS-REFERENCE TO RELATED APPLICATIONS

This application claims the benefit of U.S. Provisional Application Ser. No. 63/599,660 filed on 16 Nov. 2023 titled “MICRO RNA BIOMARKERS FOR THE DIAGNOSIS OF USHER SYNDROME”, which is hereby incorporated by reference in its entirety.

### TECHNICAL FIELD

The present disclosure relates generally to biomarkers and related methods and kits for use in the diagnosis and typing of Usher Syndrome.

### BACKGROUND

Usher syndrome (USH) represents an autosomal recessive inherited disorder that profoundly impacts hearing, vision, and balance. It is characterized by the triad of sensorineural hearing loss (SNHL), vision impairment due to retinitis pigmentosa (RP), and vestibular dysfunction. The prevalence of USH ranges from 4 to 17 cases per 100,000 individuals. USH is clinically categorized into three distinct types, distinguished by the age of onset of hearing and visual impairments, the severity of hearing loss, and the presence of balance issues. USH type 1 (USH1) is marked by early-onset SNHL, RP-related vision loss, and balance problems that commence within the first decade of life. USH type 2 (USH2) is characterized by the onset of mild to moderate nonprogressive SNHL and RP between the ages of 10 and 20, accompanied by normal vestibular function. USH type 3 (USH3) involves progressive SNHL, sometimes accompanied by balance disturbances, with a variable onset time for SNHL and RP.

USH presents as a clinically and genetically diverse condition with nine confirmed causative genes. While traditionally considered a monogenic disorder, recent studies have unveiled instances of digenic inheritance. Mutations in eight genes, including MYO7A, USH1C, CDH23, PCDH23, USH1E, USH1G, USH1K, and CIB2/DFNB48, are responsible for USH1. USH2 results from mutations in USH2A, GPR98 (also known as ADGRV1), and WHRN (also known as DFNB31), while USH3 is attributed to mutations in CLRN-1, HARS, and PDZD7.

Micro RNAs (miRNAs), small single-strand non-coding RNA molecules spanning 21 to 23 nucleotides in length, play pivotal roles in post-transcriptional gene expression regulation. These miRNA molecules are integral regulators of cellular homeostasis in both normal physiological and pathological processes. Extensive research has underscored the significance of miRNAs in the visual, auditory, and vestibular systems. Specifically, the microRNA-183 family, encompassing miRNA-183, miRNA-182, and miRNA-96, is highly conserved and indispensable for the development and maturation of sensory organs. This family of miRNAs plays crucial roles in maintaining normal quantities of hair cells and neurons within the inner ear. Moreover, it is vital for the maturation of photoreceptor cells, with disruptions in the miRNA-183 family resulting in syndromic retinal degradation and substantial vestibular impairments. With a growing research interest, miRNAs have been shown to play important roles in inner ear development and hearing loss, thus may have biomarker potential with future applications in diagnostics and treatment to restore hearing function. However, defining precise roles and targets of individual miRNAs involved in neurosensory pathways in the inner ear remains a major challenge due to the inaccessibility of the human inner ear.

Thus, there exists a need in the art for the identification of miRNA biomarkers for use in the diagnosis and typing of Usher syndrome.

### SUMMARY

In an embodiment, the present disclosure is directed to methods of diagnosing Usher Syndrome in a patient. In certain embodiments, the method comprises obtaining a biological sample from the patient, analyzing expression of a plurality of micro RNA (miRNA) isolated from said biological sample, wherein the plurality of miRNA comprises at least one of hsa-miR-363-3p, hsa-miR-150-5p, hsa-miR-155-5p, hsa-miR-223-3p, has-miR-let-7a-5p, hsa-miR-142-3p, hsa-miR-96-5p, hsa-miR-182-3p, hsa-miR-183-3p, hsa-miR-28-5p, hsa-miR-16-5p, and hsa-miR-19b-3p; and diagnosing the patient with Usher Syndrome when at least one of the plurality of miRNA are differentially expressed as compared to expression of the same miRNA in a control cell line.

In an embodiment, the present disclosure is directed to methods of detecting differential expression of one or more miRNA in a patient. In certain embodiments, the method comprises obtaining a biological sample from the patient, analyzing expression of a plurality of micro RNA (miRNA) isolated from said biological sample, wherein the plurality of miRNA comprises at least one of hsa-miR-363-3p, hsa-miR-150-5p, hsa-miR-155-5p, hsa-miR-223-3p, hsa-miR-let-7a-5p, hsa-miR-142-3p, hsa-miR-96-5p, hsa-miR-182-3p, hsa-miR-183-3p, hsa-miR-28-5p, hsa-miR-16-5p, and hsa-miR-19b-3p, and detecting whether at least one of the plurality of miRNA are differentially expressed as compared to expression of the same miRNA in a control cell line.

In yet another embodiment, the present disclosure is directed to kits for detecting at least one differentially expressed miRNA in a plurality of miRNA in a biological sample. In certain embodiments, the kit comprises a biosampling device and a lysis buffer, wherein the plurality of miRNA comprises at least one of hsa-miR-363-3p, hsa-miR-150-5p, hsa-miR-155-5p, hsa-miR-223-3p, hsa-miR-let-7a-5p, hsa-miR-142-3p, hsa-miR-96-5p, hsa-miR-182-3p, hsa-miR-183-3p, hsa-miR-28-5p, hsa-miR-16-5p, and hsa-miR-19b-3p. In certain embodiments, the kit further comprises one or more probes or primers that hybridize to one or more of the plurality of miRNA.

### BRIEF DESCRIPTION OF THE DRAWINGS

FIG. 1 shows miRNAs that are differently expressed in USH phenotypes. Among the 798 miRNAs detected by microarray 92 miRNAs were differently expressed in USH phenotypes. Among these 92 miRNAs, 20 were specific to USH1, 14 to USH2, 5 to USH3, 2 to USH1 & USH2, 5 to USH1 & USH3, 10 to USH2 & USH3 and the remaining 36 were common to all USH types.

FIG. 2 is a PCA plot showing differences between miRNA expression profiles. Each point represents the miRNA profile of a sample, yellow triangles are control cell lines, blue triangles are USHI cell lines, grey triangles are USH2, and brown triangles are USH3 cell lines. Vectors labeled with miRNA's are the top 6 miRNA's contributing to variation in the first two principal components. The table indicates the results from PERMANOVA analysis, where Pr (>F) indicates significance at values less than 0.05.

FIG. 3 is a heatmap of differential miRNAs of interest from DESeq2 contrasts of genotype and phenotype. Red tiling indicates downregulated miRNA expression, green tiling indicates upregulated miRNA expression. Rows and columns were clustered by hierarchical clustering. Vertical clusters A and B show relationships between expression patterns, predominantly separating miRNAs upregulated in the control group in cluster A, and upregulated miRNAs in the Usher phenotypes are represented in cluster B. Row names in the heatmap denote the phenotype of the cell line and which replicate, as well as the genotype reported from exome sequencing.

FIG. 4 shows NanoString miRNA counts of downregulated 6 candidate miRNAs in Usher cell lines compared to controls. Statistical comparisons were made for each miRNA using a one-way ANOVA with Tukeys adjustment for multiple comparisons, with significance denoted at p<0.05. Genotypes with the same letter above the error bars are statistically the same, while differing letters indicate significant differences from one another.

FIG. 5 shows NanoString miRNA counts of upregulated 6 candidate miRNAs in Usher cell lines, compared to controls. Statistical comparisons were made for each miRNA using a one-way ANOVA with Tukeys adjustment for multiple comparisons, with significance denoted at p<0.05. Genotypes with the same letter above the error bars are statistically the same, while differing letters indicate significant differences from one another.

FIG. 6 shows quantitative analysis of 6 downregulated candidate miRNAs in Usher cell lines, compared to controls using droplet digital PCR technology. Statistical comparisons were made for each miRNA using a one-way ANOVA with Tukey's adjustment for multiple comparisons, with significance denoted at p<0.05. Genotypes with the same letter above the error bars are statistically the same, while differing letters indicate significant differences from one another. Quantitative miRNA-ddPCR shows downregulation of 4 miRNAs in Usher cell lines, compared to controls.

FIG. 7 shows quantitative analysis of 6 upregulated candidate miRNAs in Usher cell lines, compared to controls using droplet digital PCR technology. Statistical comparisons were made for each miRNA using a one-way ANOVA with Tukeys adjustment for multiple comparisons, with significance denoted at p<0.05. Genotypes with the same letter above the error bars are statistically the same, while differing letters indicate significant differences from one another.

The figures described herein form part of the specification and are included to further demonstrate certain preferred embodiments aspects of the disclosure. In some instances, some preferred embodiments can be best understood by referring to the accompanying figures in combination with the detailed description presented herein. The description and accompanying figures may highlight a certain specific example, or a certain aspect of one or more preferred embodiments. However, one skilled in the art will understand that portions of the example or aspect may be used in combination with other examples or aspects of the disclosure.

### DETAILED DESCRIPTION

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1%, and 4%. This applies regardless of the breadth of the range.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment.

Furthermore, the particular features, structures, or characteristics can be combined in any suitable manner in one or more embodiments.

The term “about,” as used herein, refers to variation in the numerical quantity that can occur, for example, through typical measuring techniques and equipment, with respect to any quantifiable variable, including, but not limited to, concentration, mass, time, volume, concentration, temperature, homology, nucleotide count, percentages (including, but not limited to percent hearing loss), etc. As used herein, the terms “about” or “approximately” when preceding a numerical value indicates the value plus or minus a range of 10% of the value. Whether or not modified by the term “about,” the claims include equivalents to the quantities.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

As used herein, the terms “hearing loss” or “deafness” are used interchangeably and refer to the condition of lacking the power of hearing or having impaired hearing. A patient who is not able to hear as well as someone with normal hearing—hearing thresholds of 20 dB or better in both ears—is said to have “hearing loss”. Hearing loss may be mild, moderate, severe, or profound. It can affect one ear or both ears, and leads to difficulty in hearing conversational speech or loud sounds. A “deaf” patient is one who has profound hearing loss, which implies very little or no hearing.

As used herein, a “control” is an alternative sample used in an experiment for comparison purposes. A control can be “positive” or “negative.” A “control cell line” refers to a cell line originating from a control or reference sample. In certain embodiments, the control cell line is an immortalized cell line derived from a subject without Usher syndrome.

As used herein, the term “detecting” refers to determining the presence of one or more differentially expressed miRNAs of interest in a sample. Detection does not require the method to provide 100% sensitivity.

As used herein, the term “differentially expressed” or “differential expression” refers a statistically significant upregulation or downregulation of the expression of one or more miRNAs of interest in a sample as compared to the expression level of the same miRNA(s) in a control sample.

As used herein, the terms “extraction” or “isolation” refer to any action taken to separate nucleic acids from other cellular material present in the sample. The term extraction or isolation includes mechanical or chemical lysis, addition of detergent or protease, or precipitation and removal of other cellular material.

As used herein, the term “gene” refers to a DNA sequence that comprises regulatory and coding sequences necessary for the production of an RNA, which may have a non-coding function (e.g., a ribosomal or transfer RNA) or which may include a protein, polypeptide or a polypeptide precursor. The RNA or polypeptide may be encoded by a full-length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, the term “genotype” refers to the genetic makeup of an individual cell, cell culture, tissue, organism (e.g., a human), or group of organisms.

As used herein, the term “hybridize” refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid or ribonucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, “oligonucleotide” refers to a molecule that has a sequence of nucleic acid bases on a backbone comprised mainly of identical monomer units at defined intervals.

The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides that function as primers or probes are generally at least about 10-15 nucleotides in length or up to about 70, 100, 110, 150 or 200 nucleotides in length, and more preferably at least about 15 to 25 nucleotides in length, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

Oligonucleotides used as primers or probes for specifically amplifying or specifically detecting a particular target nucleic acid generally are capable of specifically hybridizing to the target nucleic acid.

As used herein, the terms “patient”, “subject”, or “individual” can be an individual organism, a vertebrate, a mammal, or a human. In some embodiments, the patient, subject, or individual is a human. In some embodiments, the patient is a human infant.

As used herein, the term “phenotype” refers to the observable characteristics of an individual cell, cell culture, organism (e.g., a human), or group of organisms which results from the interaction between that individual's genetic makeup (i.e., genotype) and the environment.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

In some embodiments, primers are at least about 10, 15, 18, or 30 nucleotides in length or up to about 100, 110, 125, or 200 nucleotides in length. In some embodiments, primers are preferably between about 15 to about 60 nucleotides in length, and most preferably between about 25 to about 40 nucleotides in length. In some embodiments, primers are about 15 to about 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

As used herein, the term “primer pair” refers to a forward and reverse primer pair (i.e., a left and right primer pair) that can be used together to amplify a given region of a nucleic acid of interest.

As used herein, the term “probe” refers to a nucleic acid sequence that interacts with a target nucleic acid via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. Probes are typically at least about 10, 15, 20, 25, 30, 35, 40, 50, 60, 75, 100 nucleotides or more in length.

As used herein, the term “sample” refers to clinical samples obtained from a patient. In some embodiments, a sample is obtained from a biological source (i.e., a “biological sample”), such as tissue or bodily fluid collected from a subject. Sample sources include, but are not limited to, mucus, sputum (processed or unprocessed), bone marrow, bronchial alveolar lavage (BAL), bronchial wash (BW), blood, bodily fluids, cerebrospinal fluid (CSF), urine, plasma, serum, or tissue (e.g., biopsy material). In some embodiments, sample sources include plasma, serum, or whole blood.

As used herein, the phrase “Usher syndrome” refers to an inherited autosomal recessive genetic disease that leads to deafness, visual impairment, and variable degrees of vestibular dysfunction. Since the gene is recessive, the condition only occurs when a patient has two copies of the mutation (homozygous). A carrier of the disease will only have one copy of the mutation (heterozygous). Usher syndrome is characterized by sensorineural hearing loss, retinitis pigmentosa (RP) and in some cases, vestibular dysfunction. Usher syndrome is a clinically and genetically heterogeneous disease, accounting for about half of all cases of combined hereditary deafness-blindness. Three clinical forms of the disease have been identified (USH I, II, and III) based on the severity of the hearing impairment, the presence or absence of vestibular dysfunction, and the age of onset of the disease. Usher syndrome Type II is the most frequent clinical form accounting for approximately 50% of all Usher syndrome cases. Usher syndrome Type II is characterized by congenital hearing loss and progressive vision loss starting in adolescence or adulthood. The hearing loss ranges from mild to severe and mainly affects the ability to hear high-frequency sounds. Vision loss occurs as the light-sensing cells of the retina gradually deteriorate. Night vision loss begins first, followed by loss of the peripheral vision. With time, these blind areas enlarge and merge to produce tunnel vision. In some cases, vision is further impaired by cataracts. Many patients become legally blind in the 5^{th} decade of life. Usher syndrome Type 2A is due to a mutation in the USH2A gene and accounts for approximately 80% of all Usher syndrome Type II cases and 40% of all Usher syndrome cases.

The present technology is not to be limited in terms of the particular embodiments described in this application, which are intended as single illustrations of individual embodiments of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In some embodiments, the disclosure is directed to methods of screening, diagnosing and typing Usher Syndrome in a patient through quantification of the expression of a plurality of micro RNAs (miRNA). The differential expression of the plurality of miRNAs serve as a biomarker for Usher Syndrome and can further serve to type a patient within one of the three types of Usher Syndrome (Type 1, Type 2, Type 3) or a subtype therein.

The present disclosure is also directed to methods of detecting differential expression of a plurality of miRNA in a patient. In some embodiments, the method comprises obtaining a biological sample from the patient, analyzing expression of a plurality of micro RNA (miRNA) isolated from said biological sample, and detecting whether at least at least 1, at least 2, at least 3, at least 4, at least 5, at least 6, at least 7, at least 8, at least 9, at least 10, at least 11, or at least 12 of the plurality of miRNA are differentially expressed as compared to expression of the same miRNA in a control cell line.

In some embodiments, the control cell line is derived from a subject that does not have Usher Syndrome. In some embodiments, the control cell line is an immortalized cell line. The immortalized cell line may be derived from the lymphoblasts of a healthy subject.

In some embodiments, the plurality of miRNAs comprises at least 1, at least 2, at least 3, at least 4, at least 5, at least 6, at least 7, at least 8, at least 9, at least 10, at least 11, or all 12 of hsa-miR-363-3p, hsa-miR-150-5p, hsa-miR-155-5p, hsa-miR-223-3p, hsa-miR-let-7a-5p, hsa-miR-142-3p, hsa-miR-96-5p, hsa-miR-182-3p, hsa-miR-183-3p, hsa-miR-28-5p, hsa-miR-16-5p, or hsa-miR-19b-3p,

In some embodiments, the plurality of miRNAs can further comprise one or more of hsa-miR-222-3p, hsa-miR-424-5p, hsa-miR-503-5p, hsa-miR-1252-5p, hsa-miR-1246, hsa-miR-182-5p, hsa-miR-1205, hsa-miR-450a-5p, hsa-miR-654-5p, hsa-miR-3934-5p, hsa-miR-221-5p, hsa-miR-34a-5p, hsa-let-7 d-5p, hsa-miR-483-3p, hsa-miR-146b-5p, hsa-miR-5010-3p, hsa-miR-591, hsa-let-7i-5p, hsa-miR-194-5p, hsa-miR-28-3p, hsa-miR-10a-5p, hsa-miR-146a-5p, hsa-miR-337-3p, hsa-miR-200c-3p, hsa-miR-5196-3p, hsa-miR-6732-3p, hsa-let-7f-5p, hsa-miR-577, hsa-let-7 g-5p, hsa-miR-183-5p, hsa-let-7b-5p, hsa-miR-148a-3p, hsa-miR-98-5p, hsa-miR-151a-5p, hsa-miR-129-2-3p, hsa-miR-519d-3p, hsa-miR-4431, hsa-miR-345-5p, hsa-miR-152-3p, hsa-miR-1260b, hsa-miR-3916, hsa-miR-4455, hsa-miR-3195, hsa-miR-195-5p, hsa-miR-1226-3p, hsa-miR-151a-3p, hsa-miR-181b-2-3p, hsa-miR-1287-5p, hsa-miR-324-5p, hsa-miR-484, hsa-miR-331-3p, hsa-miR-26b-5p, hsa-miR-513b-5p, hsa-miR-137, hsa-miR-370-3p, hsa-miR-503-3p, hsa-miR-767-5p, hsa-miR-132-3p, hsa-let-7c-5p, hsa-miR-1304-5p, hsa-miR-514a-3p, hsa-miR-342-3p, hsa-miR-181a-5p, hsa-miR-181 c-5p, hsa-miR-1193, hsa-miR-125a-3p, hsa-miR-494-3p, hsa-miR-125b-5p, hsa-miR-4284, hsa-miR-181a-2-3p, hsa-miR-551b-3p, hsa-miR-299-3p, hsa-miR-371b-5p, hsa-miR-601, hsa-miR-1827, hsa-miR-27a-3p, has-miR-582-5p, hsa-miR-574-3p, hsa-miR-1183, has-miR-542-5p, hsa-let-7 e-5p, hsa-miR-23c, hsa-miR-221-3p, hsa-miR-374b-5p, or hsa-miR-23a-3p.

In some embodiments, a method of diagnosing Usher syndrome in a patient comprises obtaining a biological sample from the patient, analyzing expression of a plurality of micro RNA (miRNA) isolated from said biological sample, and diagnosing the patient with Usher Syndrome when at least at least 1, at least 2, at least 3, at least 4, at least 5, at least 6, at least 7, at least 8, at least 9, at least 10, at least 11, or at least 12 of the plurality of miRNA are differentially expressed as compared to expression of the same miRNA in a control cell line.

In some embodiments, differential expression comprises an upregulation of the miRNA as compared to expression of the same miRNA in a control cell line. In certain embodiments, the miRNA of interest is upregulated at least 1 times, at least 1.5 times, at least 2 times, at least 2.5 times, at least 3 times, at least 4 times, at least 5 times, at least 6 times, or at least 6.5 times compared to expression of the same miRNA in a control cell line.

In some embodiments, the patient is diagnosed with Usher Syndrome when at least 1, at least 2, at least 3, at least 4, at least 5, or all 6 of hsa-miR-363-3p, hsa-miR-150-5p, hsa-miR-155-5p, hsa-miR-223-3p, hsa-miR-let-7a-5p, or hsa-miR-142-3p is upregulated as compared to expression of the same miRNA in a control cell line.

In some embodiments, the patient is diagnosed with Usher Syndrome Type 2A or Usher Syndrome Type 3A when there is an upregulation of hsa-miR-223-3p as compared to expression of the same miRNA in a control cell line.

In some embodiments, the patient is diagnosed with Usher Syndrome Type 2A when there is an upregulation of has-miR-150-5p as compared to expression of the same miRNA in a control cell line.

In some embodiments, the patient is diagnosed with Usher Syndrome Type 1B, Usher Syndrome Type 2A, or Usher Syndrome Type 3A when there is an upregulation of hsa-miR-let-7a-5p as compared to expression of the same miRNA in a control cell line.

In some embodiments, differential expression comprises a downregulation of the miRNA as compared to expression of the same miRNA in a control cell line. In certain embodiments, the miRNA of interest is downregulated at least 1 times, at least 1.5 times, at least 2 times, at least 2.5 times, at least 3 times, at least 4 times, at least 5 times, at least 6 times, or at least 6.5 times compared to expression of the same miRNA in a control cell line.

In some embodiments, the patient is diagnosed with Usher Syndrome when at least 1, at least 2, at least 3, at least 4, at least 5, or all 6 of hsa-miR-96-5p, hsa-miR-182-3p, hsa-miR-183-3p, hsa-miR-28-5p, hsa-miR-16-5p, or hsa-miR-19b-3p is downregulated as compared to expression of the same miRNA in a control cell line.

Analysis of the expression of miRNA can be performed using microarray analysis, digital droplet PCR (ddPCR), or other methods of quantifying nucleic acids.

In some embodiments, the biological sample is plasma, serum, or whole blood. In other embodiments, the biological sample is dried plasma, dried serum, and/or dried whole blood. In one embodiment, the biological sample is a dried human biological sample. In another embodiment, the biological sample is obtained from a patient having or suspected of having a hearing loss disorder (e.g., Usher syndrome, Pendred syndrome, Jervell syndrome, or Lange-Nielsen syndrome). In some embodiments, the patient is an infant patient. For example, the disclosed methods may be desirable for efficiently and non-invasively screening infant patients for Usher syndrome. In some embodiments, detecting differential expression of at least 1, at least 2, at least 3, at least 4, at least 5, at least 6, at least 7, at least 8, at least 9, at least 10, at least 11, or at least 12 of the miRNAs from the plurality of miRNA indicates an increased likelihood of the patient having Usher Syndrome. In certain embodiments, the increased likelihood of Usher Syndrome is an increase of at least about 0.5%, 1%, 5%, 10%, 15%, 20%, 25%, 30%, 35%, 40%, 45%, 50%, 55%, 60%, 65%, 70%, 75%, 80%, 85%, 90%, or 95%.

In some embodiments, the patient is at an increased likelihood of having Usher Syndrome Type 2A or Usher Syndrome Type 3A when there is an upregulation of hsa-miR-223-3p as compared to expression of the same miRNA in a control cell line. In some embodiments, the patient is at an increased likelihood of having Usher Syndrome Type 2A when there is an upregulation of has-miR-150-5p as compared to expression of the same miRNA in a control cell line. In some embodiments, the patient is at an increased likelihood of having Syndrome Type 1B, Usher Syndrome Type 2A, or Usher Syndrome Type 3A when there is an upregulation of hsa-miR-let-7a-5p as compared to expression of the same miRNA in a control cell line. In certain embodiments, the increased likelihood is an increase of at least about 0.5%, 1%, 5%, 10%, 15%, 20%, 25%, 30%, 35%, 40%, 45%, 50%, 55%, 60%, 65%, 70%, 75%, 80%, 85%, 90%, or 95%.

In some embodiments, kits for use in practicing the methods described herein are contemplated. In some embodiments, kits comprise the plurality of miRNA panel and all solutions, buffers, and vessels sufficient for performing the methods described herein. In some embodiments, the kit comprises a biosampling device and a lysis buffer, wherein the plurality of miRNA comprises hsa-miR-363-3p, hsa-miR-150-5p, hsa-miR-155-5p, hsa-miR-223-3p, hsa-miR-let-7a-5p, hsa-miR-142-3p, hsa-miR-96-5p, hsa-miR-182-3p, hsa-miR-183-3p, hsa-miR-28-5p, hsa-miR-16-5p, and/or hsa-miR-19b-3p. In an embodiment, the plurality of miRNA further comprises one or more of hsa-miR-222-3p, hsa-miR-424-5p, hsa-miR-503-5p, hsa-miR-1252-5p, hsa-miR-1246, hsa-miR-182-5p, hsa-miR-1205, hsa-miR-450a-5p, hsa-miR-654-5p, hsa-miR-3934-5p, hsa-miR-221-5p, hsa-miR-34a-5p, hsa-let-7 d-5p, hsa-miR-483-3p, hsa-miR-146b-5p, hsa-miR-5010-3p, hsa-miR-591, hsa-let-7i-5p, hsa-miR-194-5p, hsa-miR-28-3p, hsa-miR-10a-5p, hsa-miR-146a-5p, hsa-miR-337-3p, hsa-miR-200c-3p, hsa-miR-5196-3p, hsa-miR-6732-3p, hsa-let-7f-5p, hsa-miR-577, hsa-let-7 g-5p, hsa-miR-183-5p, hsa-let-7b-5p, hsa-miR-148a-3p, hsa-miR-98-5p, hsa-miR-151a-5p, hsa-miR-129-2-3p, hsa-miR-519d-3p, hsa-miR-4431, hsa-miR-345-5p, hsa-miR-152-3p, hsa-miR-1260b, hsa-miR-3916, hsa-miR-4455, hsa-miR-3195, hsa-miR-195-5p, hsa-miR-1226-3p, hsa-miR-151a-3p, hsa-miR-181b-2-3p, hsa-miR-1287-5p, hsa-miR-324-5p, hsa-miR-484, hsa-miR-331-3p, hsa-miR-26b-5p, hsa-miR-513b-5p, hsa-miR-137, hsa-miR-370-3p, hsa-miR-503-3p, hsa-miR-767-5p, hsa-miR-132-3p, hsa-let-7 c-5p, hsa-miR-1304-5p, hsa-miR-514a-3p, hsa-miR-342-3p, hsa-miR-181a-5p, hsa-miR-181 c-5p, hsa-miR-1193, hsa-miR-125a-3p, hsa-miR-494-3p, hsa-miR-125b-5p, hsa-miR-4284, hsa-miR-181a-2-3p, hsa-miR-551b-3p, hsa-miR-299-3p, hsa-miR-371b-5p, hsa-miR-601, hsa-miR-1827, hsa-miR-27a-3p, has-miR-582-5p, hsa-miR-574-3p, hsa-miR-1183, has-miR-542-5p, hsa-let-7 e-5p, hsa-miR-23c, hsa-miR-221-3p, hsa-miR-374b-5p, or hsa-miR-23a-3p.

The biosampling device can be any device known in the art used to collect a biological sample from a patient. In some embodiments, the biosampling device is a cup, slide, syringe, needle, swab, gauze, paper, pipette, vial, vacutainer, tube, or any other device suitable to collect a biological sample.

In some embodiments, the kit comprises one or more probes, primers, or primer pairs that hybridize to one or more regions of one or more of the plurality of miRNA. In some embodiments, the lysis buffer comprises guanidine hydrochloride, Tris.Cl, EDTA, Tween 20, and/or Triton X-100.

All publications and patent applications mentioned in the specification are indicative of the level of those skilled in the art to which this disclosure pertains.

Although the foregoing disclosure has been described in some detail by way of illustration and example for purposes of clarity of understanding, it will be obvious that certain changes and modifications may be practiced within the scope of the appended claims. Thus, many modifications and other embodiments of the disclosure will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims.

### EXAMPLES

The following examples are offered by way of illustration and not by way of limitation.

### Example 1

A study was designed to investigate miRNA expression patterns in USH cell lines derived from Usher patients and compare them with those in control cell lines with the aim to identify potential miRNA biomarkers for Usher syndrome.

### Materials and Methods

### Cell Lines

Three of the cell lines utilized in this study, namely D3741 (USH1), D3739 (USH1), and D2880 (USH3), were originally established at Boys Town National Research Hospital in Omaha, Nebraska, USA. These cell lines were created through the infection of lymphocytes obtained from individuals with Usher syndrome (USH) using Epstein-Barr virus (EBV) derived from the B95-8 cell line. Usher syndrome patients were recruited as donors from Boys Town National Research Hospital in Omaha, Nebraska, USA, and their participation in this study was contingent upon the approval of the institutional review board (IRB #99-06-X) at Boys Town National Research Hospital. Prior to blood collection, informed consent was obtained from all participating donors. As part of the control group, lymphocytes were also isolated from healthy donors and subsequently immortalized using EBV. In addition, a lymphocyte cell line corresponding to Usher syndrome type 2A (USH2A) was procured from the Coriell Institute (Catalog ID: GM09053, Camden, New Jersey). These cell lines were cultivated in RPMI 1640 medium, supplemented with 20% fetal bovine serum (FBS) and 50 μg/mL of gentamicin. Cultures were maintained in 100×20-mm tissue culture plates within a humidified atmosphere containing 5% CO2, at a temperature of 37° C.

### DNA Extraction and Shearing

Genomic DNA was extracted from cultured cells employing the QIAamp DNA Mini kit (Cat. No. 56304), following the recommended protocol provided by the manufacturer. The concentration of the purified DNA was quantified using the “Qubit dsDNA BR Assay Kit” on a Qubit 4.0 fluorometer. To attain an optimal fragment size conducive for downstream applications, the genomic DNA was sheared using a Covaris® M220 sonicator (Covaris LLC., Woburn, MA). Shearing resulted in fragments of approximately 250 bp. The effectiveness of the shearing process was confirmed through fragment size analysis using a D1000 Tapestation (P/N: 5067-5583, 5067-5582, Agilent Technologies). For each specific cell line under investigation (USH1B, USH1D, USH2A, and USH3A), 200 ng of sheared genomic DNA was prepared in a final volume of 50.0 μL of TE buffer, for subsequent targeted exon sequencing.

### Targeted Exon Sequencing

A custom exon probe panel targeting coding regions of 26 genes associated with syndromic and non-syndromic hearing loss was created using Agilent's SureDesign probe design tool (Agilent Technologies, Santa Clara CA). In total, 5740 biotinylated mRNA probes were synthesized covering the coding regions of 26 genes of interest (200.345 Kbp total length) and used in a hybridization and capture approach for enrichment of target DNA. Probes covered exonic regions including a 25 bp extension at 3′ and 5′ UTRs. Coding regions with >40 bp gaps in coverage received custom boosting and tiling strategies to ensure sufficient sequencing coverage. Genes covered in the panel include: ABHD12, ADGRV1, ARSG, CDH23, CIB2, CLRN1, ESPN, FOXI1, GJB2, GJB6, HARS2, KCNE1, KCNJ10, KCNQ1, MYO7A, PCDH15, PDZD7, SLC26A4, USH1C, USH1G, USH2A, WHRN, CEP250, and CEP78.

Libraries for the hearing loss exon capture were prepared using Agilent's SureSelect XT HS2 DNA with a Post-capture pooling protocol, as per manufacturer's instructions (P/N: G9985D, Agilent Technologies, Santa Clara CA). Briefly, 200 ng extracted DNA in a total volume of 50 μL 0.1× TE buffer, sheared to ˜250 bp with a Covaris M220 sonicator, was used as the starting input for library preparation. Fragmented DNA samples underwent enzymatic end-repair followed by a dA-tail ligation. Sequencing adapters were ligated to dA-overhang, each cell line received a unique dual indexed primer pair with unique molecular indices. An 8 cycle PCR amplification of adapter ligated libraries was performed under the following conditions; 1 Cycle: 98° C. (2 min), 8 Cycles: 98° C. (30 sec), 60° C. (30 sec), 72° C. (1 min), 1 Cycle: 72° C. (5 min). Indexed libraries were then hybridized with a biotinylated custom HL probe panel, and DNA-probe hybrids were captured using streptavidin beads (P/N: 65601, Thermo Fisher Scientific). Targeted libraries were purified using AMPure XP (P/N: A63880, Beckman Coulter Genomics) 1× bead clean up, and library quality was assessed using a D1000 High Sensitivity Tapestation assay (P/N: 5067-5585, 5067-5584, Agilent Technologies). Post-capture libraries were diluted to equimolar concentrations and pooled for sequencing on an Illumina NextSeq 550DX 300 cycle high output flow-cell, 150 bp paired end reads (P/N: 20024908, Illumina inc.). Targeted exon sequencing resulted in samples USH3A (695.41 Mbp), USH1B (712.23 Mbp), USH1D (796.93 Mbp), USH2A (767.00 Mbp), with an average of 20.04 million reads per sample.

### Variant Calling and Interpretation

Reads for each cell line were aligned and mapped to the human reference genome (GRCh38p.14) [18] using BWA and Samtools, respectively. Sequence variants were called using Sentieon's DNA pipeline for variant detection. Sequencing variant interpretation according to ACMG classification criteria were applied using VarSeq software version 2.4.0 (Golden Helix, Inc., Bozeman MT, https://www.goldenhelix.com).

RNA Extraction and miRNA Expression Assay

Briefly, total RNA was extracted from cell lines using QIAzol® reagent (cat. #79306), followed by purification using the RNeasy® mini kit (cat. #74004) protocol according to the manufacturer's instructions (QIAGEN Sciences Inc., Germantown, MD). Purified total RNA from cell lines (100 ng RNA per sample) was used as input for MicroRNA expression analysis. MicroRNA expression in Usher and control cell lines was quantified using NanoString© Human v3 miRNA assay (cat. #CSO-MIR3-12), performed on the nCounter Pro analysis system (NanoString Technology, Seattle, WA, USA). The assay detects 798 known human miRNAs, where each miRNA has specific tag sequences ligated with fluorescently barcoded reporter probes. After a hybridization period of 16 h at 65° C., these miRNA-specific barcodes were detected by the nCounter Digital Analyzer providing miRNA copy numbers. Raw count data from the miRNA assay was normalized using the NanoString quality control dashboard (NACHO version 2.0.0) package in R [24, 25]. There are no well-established “housekeeping” miRNAs, so NACHO's house-keeping predict=TRUE function was used to select the top 5 housekeeping miRNA candidates directly from the NanoString assay. The miRNA count data was normalized relative to internal positive, negative, and housekeeping predictions using a geometric mean, normalisation_method=“GEO”. The resulting normalized count table was used for downstream analysis in R.

MiRNA ddPCR

Following miRNA-panel screening by NanoString microarray, 12 miRNAs with significant differential expression patterns were selected for dd-PCR analysis. Of these, 6 were upregulated (hsa-miR-363-3p, hsa-miR-150-5p, hsa-miR-155-5p, hsa-miR-223-3p, has-miR-let-7a-5p, and hsa-miR-142-3p) in at least one Usher cell line and 6 miRNAs that were downregulated in Usher lines (hsa-miR-96-5p, hsa-miR-182-3p, hsa-miR-183-3p, hsa-miR-28, has-miR-16-5p, has-miR-19b-3p).

For absolute quantitation of miRNAs by droplet digital PCR, constant starting RNA concentration (10 ng) from each sample was first converted into cDNA by reverse transcription (RT) using miRNA specific primer sets (Life Technology Corp., CA, USA), following TaqMan™ RT-kit protocol (Applied Biosystems Cat. #4366596). The cDNA/RT reaction was carried out in a total volume of 15 ul consisting of 2 μL of RNA template (5 ng/μL), 3 μL of

$$Copies / ng R N A = ( ( \frac{ddPCR copy number}{ddPCR sample volume ( µl )} ) \times cDNA dilution factor \times cDNA stock volume ( µl ) ) \div R N A input ( ng )$$

specific miRNA primers, 0.15 μL dNTP-mix, 0.19 μL of RNAse-H, 1 μL of Multiscribe™ reverse transcriptase enzyme (50 U/μL), 1.5 μL of RT-buffer with a final reaction volume adjusted with nuclease free water (7.16 μL). RT reactions were performed on a PCR cycler (cfx1000, BioRad) with a set parameter of 16° C. for 30 m, 42° C. for 30 m, 85° C. for 5 m and an infinite hold at 4° C. The resulting cDNA products were diluted based on relative abundance of individual miRNAs. The standard ddPCR reaction was performed in a 20 μL reaction volume by adding 10 μL of Bio-Rad 2×ddPCR Supermix for probes, 2.0 μL of diluted cDNA, 1.0 μL of individual miRNA-specific primer-probe mix and reaction volume adjusted to 20 μL by nuclease free water. Droplet digital PCR was performed using Bio-Rad Automated QX200 droplet digital PCR system as previously described [26]. Micro-RNA copy numbers per 1 ng RNA was calculated using the equation given below:

A one-way ANOVA with a Tukey test for pairwise mean comparisons was used to determine differential expression between all cell lines where statistical significance was assigned at p value<0.05.

### Statistical Analysis

Normalized count data was imported into R statistical analysis software where all subsequent microarray analysis was performed. Usher-1D and Usher-1B cell lines had three replicates in the NanoString assay. Usher-2A, Usher-3A, and the control cell lines had 4 replicates each.

Differentially expressed (DE) miRNA analysis was performed using the “DESeq2” R package [19]. Venn diagrams depicting overlapping and unique miRNAs from DESeq2 differential analysis was performed using the ggvenn package26. All pairwise contrasts of phenotype (Usher-1B, Usher-1D, Usher-2A, Usher-3A, and NBT) were considered for differential miRNA expression analysis, and significance was assigned at a Benjamini-Hochberg (BH) corrected p-value less than 0.05.

Heatmaps of top differential miRNAs from pairwise genotype and phenotype contrasts were created using “ComplexHeatmap” package in R. For heatmaps, expression count data was scaled for large differences in variation using the formula z_i=((x_i−u_i))/s_i, where x_i is the count for miRNA i, u_i is the mean for miRNA i, and s_i is the standard deviation of miRNA I. Samples were grouped using hierarchical clustering according to miRNA expression patterns.

Principal component analysis (PCA) was performed using a Bray-Curtis dissimilarity matrix calculated from normalized count data, and principal components were visualized with the “microViz” package [21]. Significant differences between miRNA profiles was performed using a permutational multivariate analysis of variance (PERMANOVA) on Bray-Curtis dissimilarity distances, with the adonis2 function in R's “vegan” package. The PERMANOVA used a generalized linear model considering the effect of phenotype (model formula: Bray-Curtis dissimilarity matrix—Phenotype).

### Results

### Targeted Exon Sequencing

The genotype of all cell lines was confirmed using target exon sequencing as described above, and the sequencing results are summarized in Table 1. The cell line D3739 has a homozygous likely pathogenic G>A missense mutation in the CDH23 gene thus belonging to genotype USH1D. This genotype is consistently associated with USH1 where the variant creates a novel splice acceptor cite which results in an in-frame deletion of 51 base pairs, removing a calcium binding motif of the protein. The D3741 cell line contains a homozygous likely pathogenic G>T missense mutation in the MYO7A gene. Therefore, cell line D3741 is categorized as USH1B. This variant has been detected both as a homozygous mutation and compound heterozygous mutation. The USH1B cell line possesses a likely pathogenic homozygous missense variant in the MYO7A gene c.2905G>T (p.Glu968Asp) which is predicted to cause splice site variation in the myosin VIIA gene [33, 36, 39-47]. In cell line D2880, a homozygous variant in CLRN1 gene was detected at c.528T>G (p.Tyr176X) thus belonging to USH3A. This mutation causes a premature stop codon, truncating the clarin-1 protein. Interestingly, the USH2A cell line has a likely compound heterozygous mutation phenotype with two variants in the USH2A gene. The first variant, USH2A c.4338_4339del (p.Cys1447fs) is a frameshift variant which is predicted to cause a premature stop codon. The second heterozygous variant has yet to be functionally confirmed. It occurs in USH2A c.14787del (p.Glu4930fs) and is, computationally suspected to cause premature truncation or nonsense mediated decay in the production of usher protein.

Table 1. Exon capture sequencing variants classified using VarSeq. For each cell line, the phenotype that was clinically diagnosed was verified using NGS targeted exon sequencing where likely pathogenic mutations were documented. Chromosomal location of the mutations, the reference base call, and the sample mutation are all provided. The ACMG classification criteria is also provided as well as the Human Genome Variant Society variant nomenclature. The gene that is affected, zygosity, predicted inheritance, and known associated conditions for all likely pathogenic mutations are all provided.

Cell Line Sequencing Variants

| | | Chromosome | | | | [symbol] Classification |
|---|---|---|---|---|---|---|
| Cell Line | Phenotype | Position | Ref/Alt | Genotype | Classification | Criteria |
| D3739 | Usher-1D | [symbol] | G/A | Homozygous | Likely Pathogenic | [symbol] |
| D3741 | Usher-1B | [symbol] | G/T | Homozygous | Likely Pathogenic | [symbol] |
| D [symbol] | Usher-3A | [symbol] | A/C | Homozygous | [symbol] /Conflicting | [symbol] |
| Coriell(GM [symbol] ) | Usher-2A | [symbol] | [symbol] | Heterozygous | Pathogenic | [symbol] |
| | | [symbol] | [symbol] | Heterozygous | Pathogenic | [symbol] |

| | | Sequence | | Gene | Associated |
|---|---|---|---|---|---|
| Cell Line | HGVS [symbol] | Ontology | Gene Name | Inheritance | Conditions |
| D3739 | NM_ [symbol] | [symbol] | CDH [symbol] | Recessive | [symbol] |
| D3741 | NM_ [symbol] | [symbol] | MYO [symbol] A | Recessive | [symbol] |
| D [symbol] | NM_ [symbol] | [symbol] | CLRN [symbol] | Recessive | [symbol] |
| Coriell(GM [symbol] ) | NM_ [symbol] | frameshift | USH2A | Recessive | [symbol] |
| | NM_ [symbol] | frameshift | USH2A | Recessive | [symbol] |

[symbol]

indicates data missing or illegible when filed

miRNA Microarray Analysis

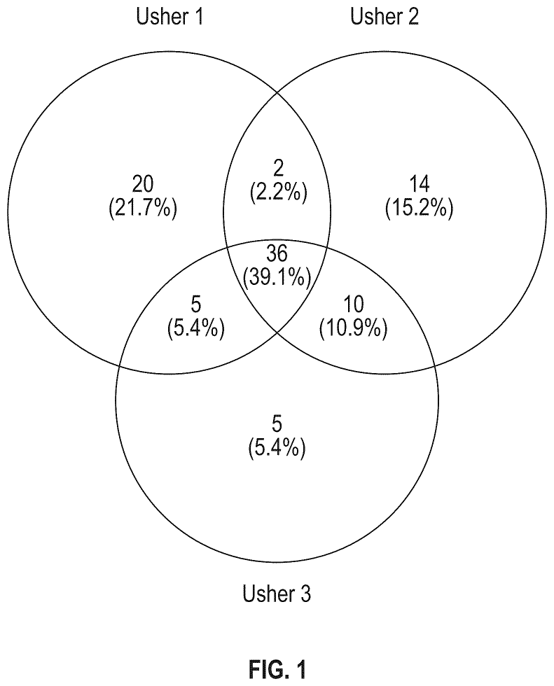

The raw data obtained from microarray were normalized as described in the “Methods” section. Normalized microarray data were further analyzed using DESeq2 to identify miRNAs that are differentially expressed in USH cell lines compared to control cells. At least a twofold increase or decrease with a BH-adjusted p value below 0.05 was considered as significantly altered miRNA expression. Using this criterion, 92 differentially expressed miRNAs were found in USH cells. Of these 92, 20 were unique to USH1, 14 to USH2, and 5 to USH3. Two were unique to both USH1 & USH2, 5 were unique to both USH1 & USH3, and 10 were unique to both USH2 & USH3. The remaining 36 were common to all USH types (FIG. 1 and Table 2).

TABLE 2
Differentially Abundant miRNAs

| Category | Total | miRNAs |
|---|---|---|
| Common To All | 36 | hsa-miR-222-3p, hsa-miR-424-5p, hsa-miR-503-5p, |
| | | hsa-miR-1252-5p, hsa-miR-1246, hsa-miR-182-5p, |
| | | hsa-miR-1205, hsa-miR-450a-5p, hsa-miR-654-5p, |
| | | hsa-miR-3934-5p, hsa-miR-221-5p, hsa-miR-34a-5p, |
| | | hsa-miR-150-5p, hsa-let-7d-5p, hsa-miR-483-3p, |
| | | hsa-miR-146b-5p, hsa-miR-5010-3p, hsa-miR-591, |
| | | hsa-miR-155-5p, hsa-miR-363-3p, hsa-let-7i-5p, |
| | | hsa-miR-194-5p, hsa-miR-28-3p, hsa-miR-10a-5p, |
| | | hsa-miR-146a-5p, hsa-miR-337-3p, hsa-miR-200c-3p, |
| | | hsa-miR-96-5p, hsa-miR-5196-3p + hsa-miR-6732-3p, hsa-let-7f-5p, |
| | | hsa-miR-577, hsa-let-7g-5p, hsa-miR-183-5p, |
| | | hsa-let-7b-5p, hsa-miR-148a-3p, hsa-miR-98-5p |
| Usher 1 Only | 20 | hsa-miR-151a-5p, hsa-miR-129-2-3p, hsa-miR-519d-3p, hsa-miR- |
| | | 4431, hsa-miR-345-5p, hsa-miR-152-3p, |
| | | hsa-miR-1260b, hsa-miR-3916, hsa-miR-4455, hsa-miR-3195, hsa-miR- |
| | | 195-5p, hsa-miR-1226-3p, |
| | | hsa-miR-151a-3p, hsa-miR-181b-2-3p, hsa-miR-1287-5p, hsa-miR-324- |
| | | 5p, hsa-miR-484, hsa-miR-16-5p, |
| | | hsa-miR-331-3p, hsa-miR-26b-5p |
| Usher 2 Only | 14 | hsa-miR-513b-5p, hsa-miR-137, hsa-miR-370-3p, hsa-miR-503-3p, hsa- |
| | | miR-767-5p, hsa-miR-132-3p, hsa-let-7c-5p, |
| | | hsa-miR-1304-5p, hsa-miR-514a-3p, hsa-let-7a-5p, hsa-miR-342- |
| | | 3p, hsa-miR-181a-5p, hsa-miR-181c-5p, hsa-miR-1193 |
| Usher 3 Only | 5 | hsa-miR-125a-3p, hsa-miR-494-3p, hsa-miR-125b-5p, hsa-miR- |
| | | 4284, hsa-miR-181a-2-3p |
| Usher 1 and 2 | 2 | hsa-miR-551b-3p, hsa-miR-299-3p |
| Usher 1 and 3 | 5 | hsa-miR-371b-5p, hsa-miR-601, hsa-miR-1827, hsa-miR-27a-3p, hsa- |
| | | miR-582-5p |
| Usher 2 and 3 | 10 | hsa-miR-574-3p, hsa-miR-223-3p, hsa-miR-1183, hsa-miR-28-5p, hsa- |
| | | miR-542-5p, hsa-let-7e-5p, hsa-miR-23c, |
| | | hsa-miR-221-3p, hsa-miR-374b-5p, hsa-miR-23a-3p |

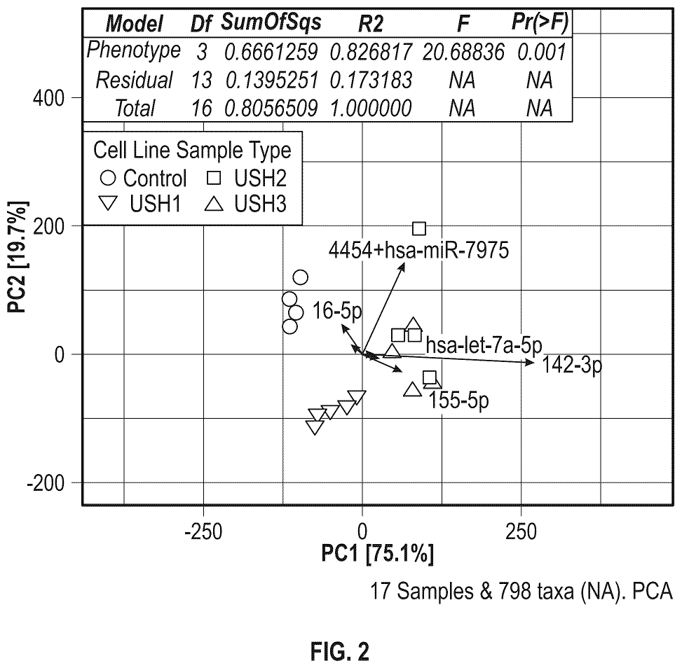

PERMANOVA analysis of miRNA microarray data confirmed that miRNA expression profiles were significantly different between all cell lines with a BH-adjusted p-value of 0.001 (FIG. 2). Results obtained using PERMANOVA were corroborated in PCA analysis, where there is distinct clustering by cell line. All Usher microRNA profiles cluster according to their respective cell lines (FIG. 2). However, both USH2A and USH3A clustered more similarly with one another than with controls or USH1 phenotypes, and a total of 94.8% of the variation in the miRNA expression profile dataset explained in the ordination of the first two principal components (FIG. 2). Additionally, of the top 6 miRNAs driving variation between samples, hsa-miR-155-5p, hsa-miR-142-3p, and hsa-let-7a-5p appear to drive differences in Usher cell lines, particularly USH2A and USH3A samples. Hsa-miR-16-5p and hsa-miR-19b-3p appear to drive variation in the direction of the control cell lines, and hsa-miR-4454+hsa-miR-7975 seems to explain variation in one of the USH2A sample replicates.

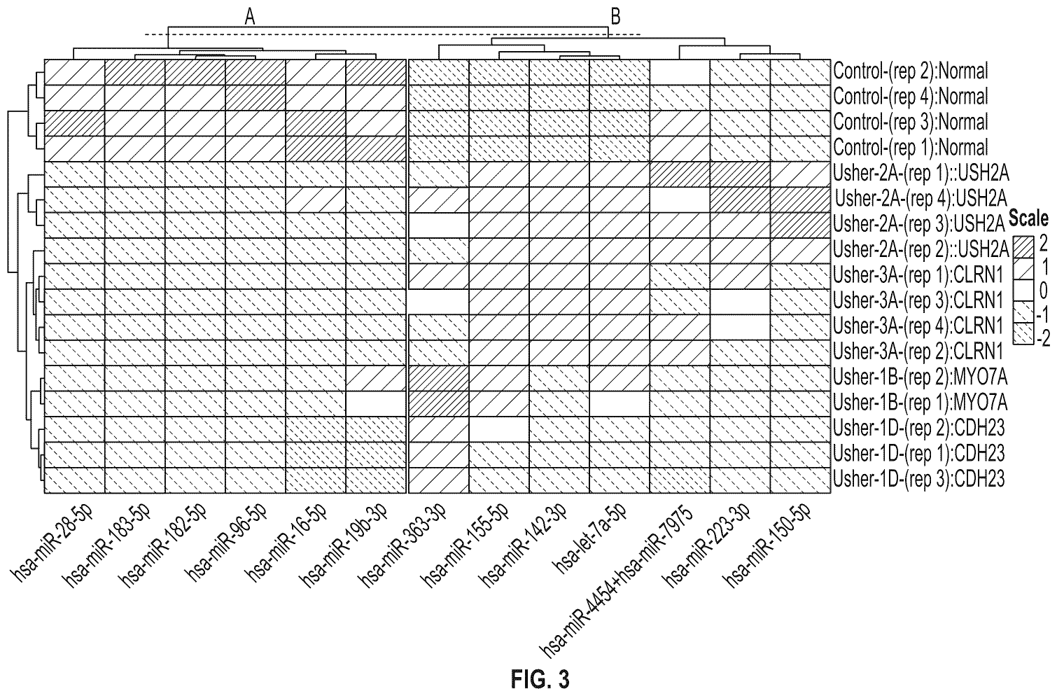

Table 3 provides a list of top 12 differentially expressed miRNAs in USH cells compared to controls. FIG. 3 is a heatmap that shows expression of those miRNAs in USH and control cell lines. All miRNAs that are included in this list had to be differentially expressed in the DESeq2 analysis, have a BH-adjusted p-value<0.05, and have at least a 2-fold change in miRNA expression between Usher cell lines and controls. These 12 miRNAs had an average copy number higher than 50. The following six miRNAs, hsa-miR-16-5p, hsa-miR-19b-3p, hsa-miR-4454+hsa-miR-7975, hsa-miR-142-3p, hsa-miR-155-5p, and hsa-let-7a-5p that were included in these 12 were among the top 6 miRNAs, explaining variation in the PCA biplot (FIG. 2).

TABLE 3

| | Usher 1 | | Usher 2 | | Usher 3 | | Mean Log2FC vs. Control | |
|---|---|---|---|---|---|---|---|---|
| | | P-Value | | P-Value | | P-Value | Average | Standard |
| microRNA | log2FC | (BH-adj.) | log2FC | (BH-adj.) | log2FC | (BH-adj.) | log2FC | Dev. |
| hsa-miR-150-5p | 4.8901 | 0.0000 | 9.7786 | 0.0000 | 6.5893 | 0.0000 | 7.0860 | 2.4818 |
| hsa-miR-155-5p | 4.1500 | 0.0000 | 4.6412 | 0.0000 | 4.6898 | 0.0000 | 4.4936 | 0.2986 |
| hsa-miR-363-3p | 4.1282 | 0.0000 | 3.3498 | 0.0000 | 3.3819 | 0.0000 | 3.6200 | 0.4404 |
| hsa-miR-223-3p | 1.6878 | 0.0002 | 4.8040 | 0.0000 | 3.2343 | 0.0000 | 3.2420 | 1.5581 |
| hsa-let-7a-5p | 0.9413 | 0.0095 | 2.0768 | 0.0000 | 1.9006 | 0.0000 | 1.6396 | 0.6111 |
| hsa-miR-142-3p | 0.5645 | 0.0259 | 1.6779 | 0.0000 | 1.5316 | 0.0000 | 1.2580 | 0.6051 |
| hsa-miR-19b-3p | −1.4389 | 0.0001 | −0.9554 | 0.0293 | −1.1786 | 0.0041 | −1.1910 | 0.2419 |
| hsa-miR-28-5p | −1.9993 | 0.0000 | −2.5731 | 0.0000 | −2.0932 | 0.0000 | −2.2219 | 0.3078 |
| hsa-miR-16-5p | −2.1845 | 0.0000 | −1.0125 | 0.0487 | −1.3422 | 0.0044 | −1.5130 | 0.6044 |
| hsa-miR-183-5p | −2.2771 | 0.0000 | −2.6795 | 0.0000 | −2.3467 | 0.0000 | −2.4344 | 0.2151 |
| hsa-miR-96-5p | −2.9386 | 0.0000 | −3.2701 | 0.0000 | −2.7408 | 0.0000 | −2.9832 | 0.2674 |
| hsa-miR-182-5p | −6.2691 | 0.0024 | −8.0376 | 0.0005 | −6.1735 | 0.0073 | −6.8267 | 1.0498 |

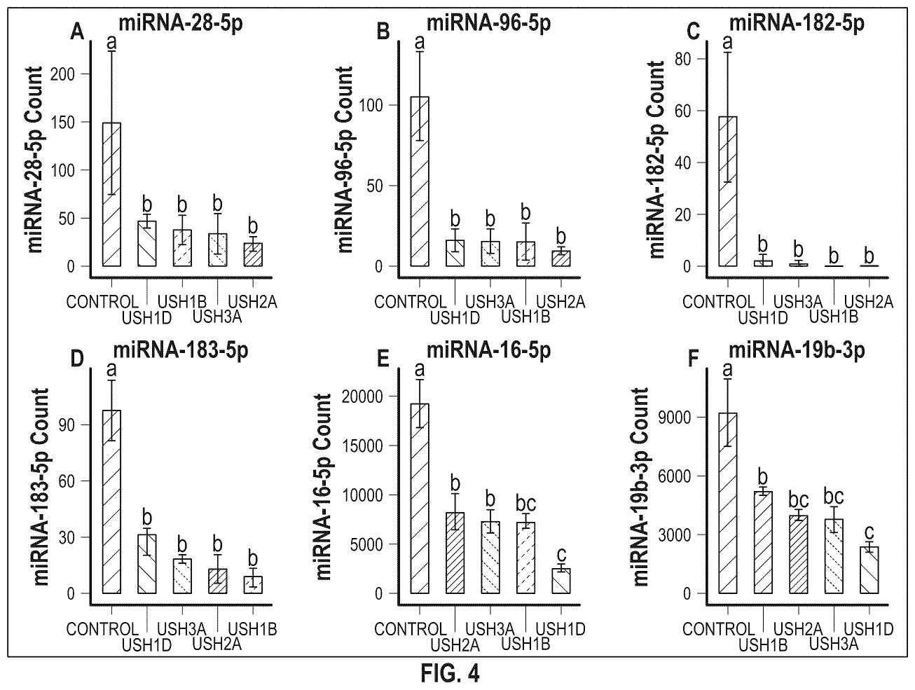

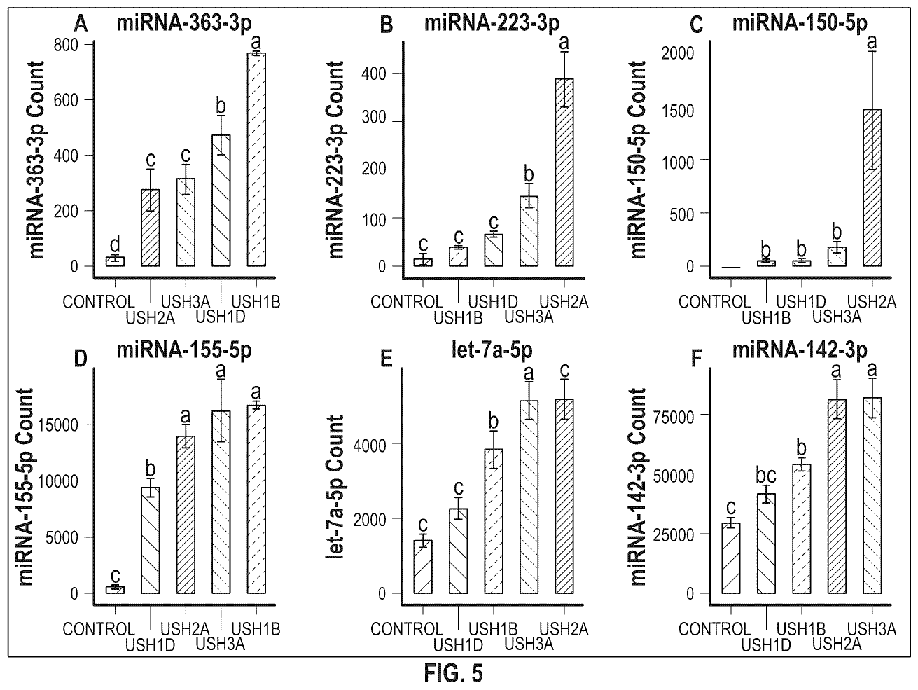

In both the PCA and heatmap USH2 and USH3 grouped together. Control samples clustered together, and USH1B and USH1D samples were most similarly aligned (FIG. 3). Among these 12 miRNAs 6 were down-regulated (FIG. 4) and 6 were up-regulated (FIG. 5). As shown in FIG. 4, miRNA-28-5p, miRNA-96-5p, miRNA-182-3p, miRNA-183-3p, miRNA-16-5-p and miRNA-19b-3p were downregulated in all USH types compared to controls. As shown in FIG. 5, panels A, D, and F, miRNA-363-3p, miRNA-155-5p and miRNA-142-3p were upregulated in all USH types. MiRNA-223-3p (FIG. 5B) is upregulated only in USH2A and USH3A. As shown in FIG. 5C, miRNA-150-5p is only upregulated in USH2A. MiRNA-let7a-5p is upregulated in USH2A, USH3A and USH1B (FIG. 5E). However, it is not changed in USH1D (FIG. 5E) showing that the same phenotype with different genotypes may have different miRNA expression patterns.

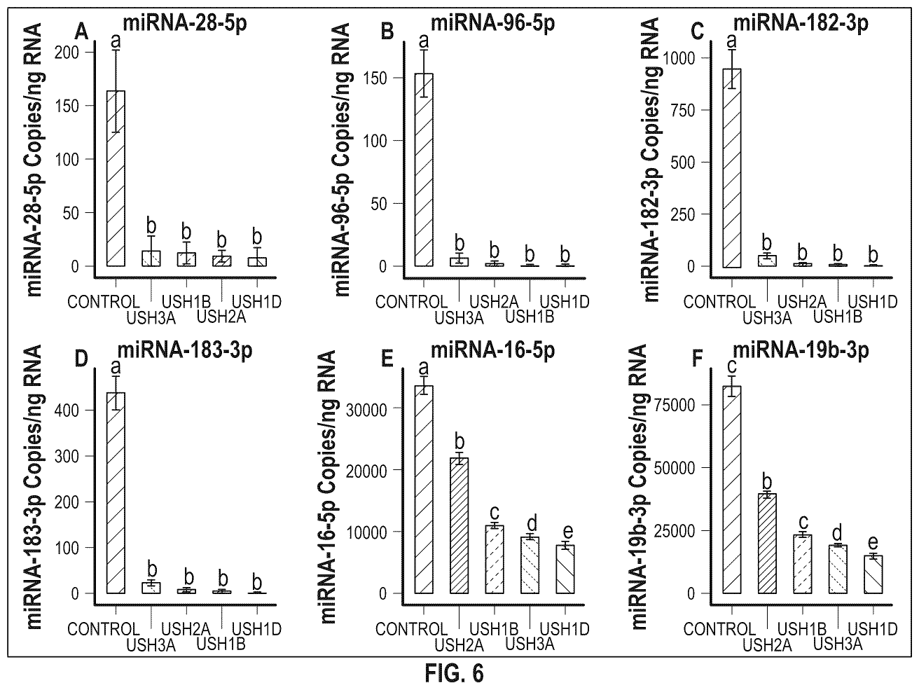

To confirm microarray data, the top 12 differentially expressed miRNAs were analyzed using droplet digital PCR technology. FIG. 6 shows the expression of 6 miRNAs that were downregulated in USH cells compared to controls. Those miRNAs include miRNA-28-5p, miRNA-96-5p, miRNA-182-3p, miRNA-183-3p, miRNA-16-5-p and miRNA-19b-3p. All 6 miRNAs showed statistically significant downregulation compared to controls. These results confirm microarray results.

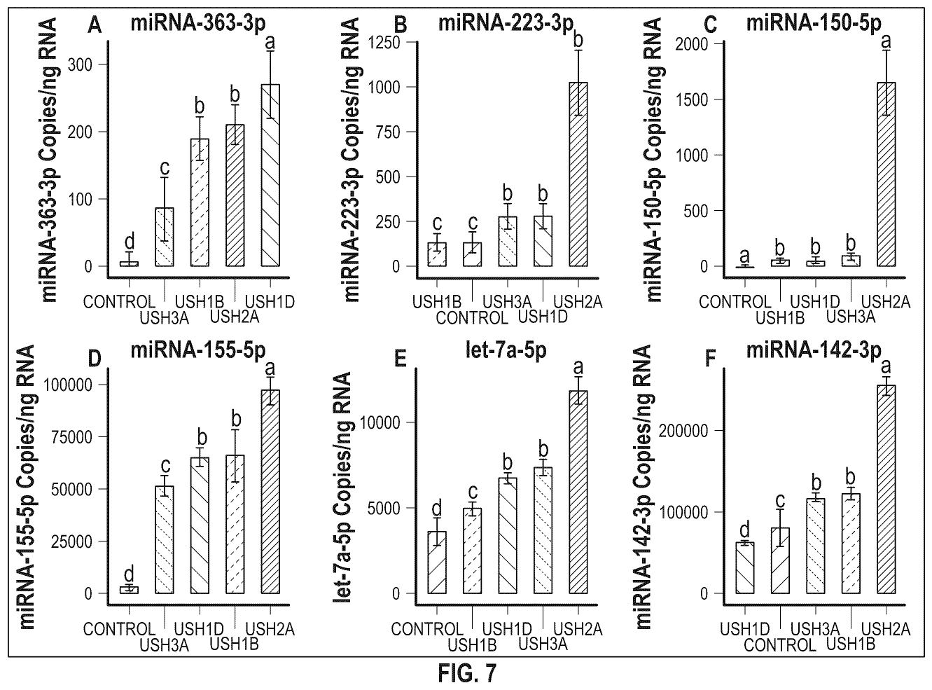

Four miRNAs, miRNA-28-5p, miRNA-96-5p, miRNA-182-3p, and miRNA-183-3p behave in a similar way. There was no statistically significant difference among USH types (FIG. 6 panels A, B, C & D). However, miRNA-16-5-p and miRNA-19b-3p showed variation within USH types. Both miRNAs showed a similar pattern, USH1D showing the highest downregulation and USH2A showing the lowest downregulation (FIG. 6 panels E & F). FIG. 7 shows the expression of 6 miRNAs that were upregulated in USH cells compared to controls. FIG. 7A shows expression results for miRNA-363-3p where all USH types show statistically significant upregulation except for USH3A. Results for miRNA-223-3p (FIG. 7B) show that all USH types show statistically significant upregulation except for USH1B. FIG. 7C shows expression results for miRNA-150-5p where only USH2A shows statistically significant upregulation. MiRNA-155-5p, miRNA-let-7a-5p, and miRNA-142-3p are upregulated in all USH types compared to control (FIG. 7, panels D, E & F). However, miRNA-223-3p was upregulated in all USH types except for USH1B (FIG. 7, panel B).

### DISCUSSION

The Usher cell lines exhibited distinct miRNA expression profiles, as illustrated in Table 2. These variations were not only evident among Usher type 1, 2, and 3 phenotypes but also discernible between USH1B and USH1D genotypes. According to microarray data presented in FIG. 2, the miRNA expressions in all Usher samples significantly differed from those in the control samples. These differences in miRNA expression may be further complicated by unique combinations of sequence variants contributing to the phenotype. For instance, in the USH2A cell line, two heterozygous frameshift mutations were identified that appear to have a compounded effect, resulting in the observed phenotype. This compound mutation effect, although somewhat unconventional, has been documented in Usher syndrome, challenging the widely accepted notion that homozygous recessive mutations exclusively underlie the disorder.

In contrast, USH1D, USH1B, and USH3A all exhibited homozygous recessive mutations, aligning with the classical definition of autosomal recessive inheritance in Usher patients.

In this study, the expression profiles of 798 miRNAs were assessed across different cell line types, revealing intriguing variations. Notably, several miRNAs exhibited distinct patterns that remained consistent across various analytical methods and assays, some of which have been previously reported in the literature.

The miRNA-183/182/96 family emerged as a key player, showing significant downregulation in all Usher cell lines compared to controls, as evident from both DESeq2 analysis of microarray results (FIG. 4 panels B, C &D) and ddPCR results (FIG. 6 panels B, C & D). Previous research showcased the significance of these miRNAs, as their inactivation in mice led to significant developmental issues in cochlear hair cells. Moreover, mutations affecting miRNA-96-5p have been linked to impaired growth, maturation, and, in some cases, malformation of cochlear hair cells in mice and humans. Additionally, alterations in the expression of the microRNA-183/182/96 family have been associated with retinal dystrophy, a component of Usher syndrome. The findings of the present study align with existing literature, supporting the notion that microRNA-183/182/96 downregulation can serve as a diagnostic biomarker for Usher syndrome.

Notably, the microarray data revealed an average 2.43±0.22-fold decrease in miRNA-183-3p expression, a 2.98±0.27-fold decrease in miRNA-96-5p expression, and approximately a 6.83±1.05-fold decrease in miRNA-182-5p expression in Usher samples compared to controls (FIG. 2 & Table 2). This downregulation across the entire miR-183/182/96 family was confirmed using ddPCR (FIG. 6 panels B, C & D) where Tukey HSD post hoc tests indicated significantly higher expression of all three miRNAs in control samples compared to Usher lines, with no significant differences observed among Usher lines (FIG. 6 panels B, C & D).

Another noteworthy finding was the significant downregulation of miRNA-28-5p in all Usher samples, with an average 2.22±0.31-fold lower expression compared to control samples (FIG. 3 & Table 2). Although no prior associations between miRNA-28-5p and Usher syndrome have been reported, a prior study suggested that miRNA-28 may target and regulate the expression of the cone-rod homeobox gene (CRX), making it a potential candidate for retinal degeneration, a component of Usher syndrome.

MiRNA-16-5p emerged as a driver of variation, particularly toward control samples, displaying an average 1.51±0.60-fold higher expression in control samples compared to Usher lines (FIG. 2 & Table 2). Interestingly, some literature links elevated miRNA-16-5p expression to noise-induced hearing loss (NIHL). However, surprisingly, the present study indicated low levels of miRNA-16-5p in Usher samples compared to controls, suggesting that the hearing loss cause by Usher and NIHL may have different mechanisms. Additionally, miRNA-19b-3p and miRNA-16-5p which exhibited a downregulation, have little known relevance to Usher syndrome. However, elevated levels of both miR-16-5p and miR-19b-3p have been shown to mitigate injury from amyloid R peptide insult associated with Alzheimer's disease.

Six miRNAs, namely miRNA-150-5p, miRNA-155-5p, miRNA-363-3p, miRNA-223-3p, miRNA-let-7a-5p, and miRNA-142-3p, exhibited upregulation in the Usher cell lines compared to the control (Table 2 & FIG. 7). Among these, miRNA-155-5p, miRNA-142-3p, and miRNA-let-7a-5p were among the most abundant miRNAs in the dataset. Notably, miRNA-142-3p made up approximately 37.3% of miRNA copies in Usher samples, compared to 18.5% in the control line. This miRNA displayed an average 1.26-fold higher expression in Usher samples and holds potential as a robust biomarker candidate. While miRNA-142-3p has not been previously associated with Usher or sensorineural hearing loss (SNHL), its downregulation has been observed in the aqueous humor of patients with central retinal vein occlusion (CRVO), a condition occasionally linked to retinitis pigmentosa (RP). Interestingly, the present study found miRNA-142-3p to be one of the most abundant miRNAs in all samples, particularly in Usher lines (˜40% of all miRNA copies), which contrasts with observations in CRVO.

Additionally, miRNA-Let-7a emerged as a prominent driver of variation in both PCA and DESeq2 analyses, exhibiting an average 1.6±0.61-fold increase compared to control samples (FIG. 2 & Table 2). In Usher samples, miRNA-Let-7a accounted for 2.35% of the total miRNA copies per genotype, while control samples displayed only 0.9%. Analysis of the heatmap revealed that both USH2A and USH3A exhibited the highest levels of miRNA-Let-7a and miRNA-142-3p, while USH1 genotypes displayed lower expression (FIG. 3), especially USH1D. This observation is consistent with the trajectory of the miR-Let-7a and miR-142-3p vectors in the PCA biplot (FIG. 2).

In addition to examining miRNA expression patterns, targeted exome sequencing was conducted to identify genotypes and likely pathogenic sequence variants in previously clinically diagnosed Usher patient-derived cell lines (Table 1). Furthermore, microarray observations were validated with ddPCR quantification of the 12 miRNAs, obtaining consistent results when compared to microarray data. These 12 miRNAs, characterized by their consistency across statistical tests and assays (ddPCR and microarray), present a list of potential biomarkers present in lymphocytes that could aid in the diagnosis and typing of Usher Syndrome.

In summary, the miRNAs identified by the present study offer a promising and novel set of candidates for Usher syndrome diagnostics.

What is claimed is:

1. A method of screening a patient for Usher Syndrome, the method comprising:
obtaining a biological sample from the patient;
analyzing expression of a plurality of micro RNA (miRNA) isolated from said biological sample, wherein the plurality of miRNA comprises one or more of hsa-miR-363-3p, hsa-miR-150-5p, hsa-miR-155-5p, hsa-miR-223-3p, has-miR-let-7a-5p, hsa-miR-142-3p, hsa-miR-96-5p, hsa-miR-182-3p, hsa-miR-183-3p, hsa-miR-28-5p, hsa-miR-16-5p, and hsa-miR-19b-3p; and
diagnosing the patient with Usher Syndrome when at least one of the plurality of miRNA are differentially expressed as compared to expression of the same miRNA in a control cell line.

2. The method of claim 1, wherein the plurality of miRNA comprises three or more of hsa-miR-363-3p, hsa-miR-150-5p, hsa-miR-155-5p, hsa-miR-223-3p, has-miR-let-7a-5p, hsa-miR-142-3p, hsa-miR-96-5p, hsa-miR-182-3p, hsa-miR-183-3p, hsa-miR-28-5p, hsa-miR-16-5p, and hsa-miR-19b-3p.

3. The method of claim 2, wherein the plurality of miRNA comprises six or more of hsa-miR-363-3p, hsa-miR-150-5p, hsa-miR-155-5p, hsa-miR-223-3p, has-miR-let-7a-5p, hsa-miR-142-3p, hsa-miR-96-5p, hsa-miR-182-3p, hsa-miR-183-3p, hsa-miR-28-5p, hsa-miR-16-5p, and hsa-miR-19b-3p.

4. The method of claim 1, wherein said differential expression comprises an upregulation or downregulation of the miRNA.

5. The method of claim 1, wherein the patient is diagnosed with Usher Syndrome when at least one of hsa-miR-363-3p, hsa-miR-150-5p, hsa-miR-155-5p, hsa-miR-223-3p, hsa-miR-let-7a-5p, or hsa-miR-142-3p is upregulated as compared to expression of the same miRNA in a control cell line.

6. The method of claim 5, wherein at least three of hsa-miR-363-3p, hsa-miR-150-5p, hsa-miR-155-5p, hsa-miR-223-3p, hsa-miR-let-7a-5p, or hsa-miR-142-3p are upregulated as compared to expression of the same miRNA in a control cell line.

7. The method of claim 1, wherein the patient is diagnosed with Usher Syndrome when at least one of hsa-miR-96-5p, hsa-miR-182-3p, hsa-miR-183-3p, hsa-miR-28-5p, hsa-miR-16-5p, or hsa-miR-19b-3p is downregulated as compared to expression of the same miRNA in a control cell line.

8. The method of claim 7, wherein at least three of hsa-miR-96-5p, hsa-miR-182-3p, hsa-miR-183-3p, hsa-miR-28-5p, hsa-miR-16-5p, or hsa-miR-19b-3p are downregulated as compared to expression of the same miRNA in a control cell line.

9. The method of claim 4, further comprising diagnosing the patient with Usher Syndrome Type 2A or Usher Syndrome Type 3A when there is an upregulation of hsa-miR-223-3p as compared to expression of the same miRNA in a control cell line, or Usher Syndrome Type 2A when there is an upregulation of has-miR-150-5p as compared to expression of the same miRNA in a control cell line, or Usher Syndrome Type 1B, Usher Syndrome Type 2A, or Usher Syndrome Type 3A when there is an upregulation of hsa-miR-let-7a-5p as compared to expression of the same miRNA in a control cell line.

10. The method of claim 1, wherein the plurality of miRNA further comprises one or more of hsa-miR-222-3p, hsa-miR-424-5p, hsa-miR-503-5p, hsa-miR-1252-5p, hsa-miR-1246, hsa-miR-182-5p, hsa-miR-1205, hsa-miR-450a-5p, hsa-miR-654-5p, hsa-miR-3934-5p, hsa-miR-221-5p, hsa-miR-34a-5p, hsa-let-7 d-5p, hsa-miR-483-3p, hsa-miR-146b-5p, hsa-miR-5010-3p, hsa-miR-591, hsa-let-7i-5p, hsa-miR-194-5p, hsa-miR-28-3p, hsa-miR-10a-5p, hsa-miR-146a-5p, hsa-miR-337-3p, hsa-miR-200c-3p, hsa-miR-5196-3p, hsa-miR-6732-3p, hsa-let-7f-5p, hsa-miR-577, hsa-let-7 g-5p, hsa-miR-183-5p, hsa-let-7b-5p, hsa-miR-148a-3p, hsa-miR-98-5p, hsa-miR-151a-5p, hsa-miR-129-2-3p, hsa-miR-519d-3p, hsa-miR-4431, hsa-miR-345-5p, hsa-miR-152-3p, hsa-miR-1260b, hsa-miR-3916, hsa-miR-4455, hsa-miR-3195, hsa-miR-195-5p, hsa-miR-1226-3p, hsa-miR-151a-3p, hsa-miR-181b-2-3p, hsa-miR-1287-5p, hsa-miR-324-5p, hsa-miR-484, hsa-miR-331-3p, hsa-miR-26b-5p, hsa-miR-513b-5p, hsa-miR-137, hsa-miR-370-3p, hsa-miR-503-3p, hsa-miR-767-5p, hsa-miR-132-3p, hsa-let-7 c-5p, hsa-miR-1304-5p, hsa-miR-514a-3p, hsa-miR-342-3p, hsa-miR-181a-5p, hsa-miR-181 c-5p, hsa-miR-1193, hsa-miR-125a-3p, hsa-miR-494-3p, hsa-miR-125b-5p, hsa-miR-4284, hsa-miR-181a-2-3p, hsa-miR-551b-3p, hsa-miR-299-3p, hsa-miR-371b-5p, hsa-miR-601, hsa-miR-1827, hsa-miR-27a-3p, has-miR-582-5p, hsa-miR-574-3p, hsa-miR-1183, has-miR-542-5p, hsa-let-7 e-5p, hsa-miR-23c, hsa-miR-221-3p, hsa-miR-374b-5p, or hsa-miR-23a-3p.

11. The method of claim 1, wherein said analyzing step is performed using microarray analysis or digital droplet PCR (ddPCR).

12. The method of claim 1, wherein the biological sample is plasma, serum, or whole blood.

13. The method of 1, wherein the biological sample is from a patient having or suspected of having a hearing loss disorder.

14. The method of claim 1, wherein the patient is a human infant.

15. The method of claim 1, wherein the control cell line is an immortalized cell line derived from a subject without Usher Syndrome.

16. A kit for detecting at least one differentially expressed micro RNA (miRNA) in a plurality of miRNA in a biological sample comprising:
a biosampling device; and
a lysis buffer,
wherein the plurality of miRNA comprises one or more of hsa-miR-363-3p, hsa-miR-150-5p, hsa-miR-155-5p, hsa-miR-223-3p, hsa-miR-let-7a-5p, hsa-miR-142-3p, hsa-miR-96-5p, hsa-miR-182-3p, hsa-miR-183-3p, hsa-miR-28-5p, hsa-miR-16-5p, and hsa-miR-19b-3p.

17. The kit of claim 16, wherein the plurality of miRNA comprises three or more of hsa-miR-363-3p, hsa-miR-150-5p, hsa-miR-155-5p, hsa-miR-223-3p, has-miR-let-7a-5p, hsa-miR-142-3p, hsa-miR-96-5p, hsa-miR-182-3p, hsa-miR-183-3p, hsa-miR-28-5p, hsa-miR-16-5p, and hsa-miR-19b-3p.

18. The kit of claim 17, wherein the plurality of miRNA comprises six or more of hsa-miR-363-3p, hsa-miR-150-5p, hsa-miR-155-5p, hsa-miR-223-3p, has-miR-let-7a-5p, hsa-miR-142-3p, hsa-miR-96-5p, hsa-miR-182-3p, hsa-miR-183-3p, hsa-miR-28-5p, hsa-miR-16-5p, and hsa-miR-19b-3p.

19. The kit of claim 16, wherein the plurality of miRNA further comprises one or more of hsa-miR-222-3p, hsa-miR-424-5p, hsa-miR-503-5p, hsa-miR-1252-5p, hsa-miR-1246, hsa-miR-182-5p, hsa-miR-1205, hsa-miR-450a-5p, hsa-miR-654-5p, hsa-miR-3934-5p, hsa-miR-221-5p, hsa-miR-34a-5p, hsa-let-7 d-5p, hsa-miR-483-3p, hsa-miR-146b-5p, hsa-miR-5010-3p, hsa-miR-591, hsa-let-7i-5p, hsa-miR-194-5p, hsa-miR-28-3p, hsa-miR-10a-5p, hsa-miR-146a-5p, hsa-miR-337-3p, hsa-miR-200c-3p, hsa-miR-5196-3p, hsa-miR-6732-3p, hsa-let-7f-5p, hsa-miR-577, hsa-let-7 g-5p, hsa-miR-183-5p, hsa-let-7b-5p, hsa-miR-148a-3p, hsa-miR-98-5p, hsa-miR-151a-5p, hsa-miR-129-2-3p, hsa-miR-519d-3p, hsa-miR-4431, hsa-miR-345-5p, hsa-miR-152-3p, hsa-miR-1260b, hsa-miR-3916, hsa-miR-4455, hsa-miR-3195, hsa-miR-195-5p, hsa-miR-1226-3p, hsa-miR-151a-3p, hsa-miR-181b-2-3p, hsa-miR-1287-5p, hsa-miR-324-5p, hsa-miR-484, hsa-miR-331-3p, hsa-miR-26b-5p, hsa-miR-513b-5p, hsa-miR-137, hsa-miR-370-3p, hsa-miR-503-3p, hsa-miR-767-5p, hsa-miR-132-3p, hsa-let-7 c-5p, hsa-miR-1304-5p, hsa-miR-514a-3p, hsa-miR-342-3p, hsa-miR-181a-5p, hsa-miR-181 c-5p, hsa-miR-1193, hsa-miR-125a-3p, hsa-miR-494-3p, hsa-miR-125b-5p, hsa-miR-4284, hsa-miR-181a-2-3p, hsa-miR-551b-3p, hsa-miR-299-3p, hsa-miR-371b-5p, hsa-miR-601, hsa-miR-1827, hsa-miR-27a-3p, has-miR-582-5p, hsa-miR-574-3p, hsa-miR-1183, has-miR-542-5p, hsa-let-7 e-5p, hsa-miR-23c, hsa-miR-221-3p, hsa-miR-374b-5p, or hsa-miR-23a-3p.

20. The kit of claim 16, further comprising one or more probes or primers that hybridize to one or more of the plurality of miRNA.
